(19) United States
(12) Patent Application Publication
(10) Pub. No.: US 2026/0114843 A1
(43) Pub. Date: Apr. 30, 2026

(54) ULTRASOUND DIAGNOSTIC APPARATUS AND METHOD OF CONTROLLING ULTRASOUND DIAGNOSTIC APPARATUS

(71) Applicant: FUJIFILM Corporation, Tokyo (JP)

(72) Inventor: Riki IGARASHI, Kanagawa (JP)

(73) Assignee: FUJIFILM Corporation, Tokyo (JP)

(21) Appl. No.: 19/369,335

(22) Filed: Oct. 27, 2025

(30) Foreign Application Priority Data
Oct. 30, 2024 (JP) 2024-190566

(51) Int. Cl. A61B 8/08; A61B 8/00; G06T 7/12; G06T 7/149

(52) U.S. Cl. CPC A61B 8/0883; A61B 8/5215; G06T 7/12; G06T 7/149; G06T 2207/10132; G06T 2207/20076; G06T 2207/20104; G06T 2207/20116; G06T 2207/30048

(57) ABSTRACT

Provided are an ultrasound diagnostic apparatus that can easily and accurately correct a contour of an intracardiac cavity and a method of controlling the ultrasound diagnostic apparatus. An ultrasound diagnostic apparatus includes an intracardiac cavity extraction unit that extracts an intracardiac cavity from an ultrasound image in which a heart of a subject is imaged; a correction possibility estimation unit that analyzes a contour of the extracted intracardiac cavity and estimates a possibility of correction in each portion of the contour; a control point specifying unit that specifies a control point on the contour based on the estimated possibility of correction; an input device for a user to perform an input operation; a correction operation reception unit that receives a correction operation performed by the user via the input device on the specified control point; and a contour correction unit that corrects the contour based on the received correction operation.

### CROSS-REFERENCE TO RELATED APPLICATIONS

The present application claims priority under 35 U.S.C. § 119 to Japanese Patent Application No. 2024-190566, filed on October 30, 2024. The above application is hereby expressly incorporated by reference, in its entirety, into the present application.

### BACKGROUND OF THE INVENTION

### 1. Field of the Invention

The present invention relates to an ultrasound diagnostic apparatus that captures an ultrasound image of a heart of a subject and a method of controlling the ultrasound diagnostic apparatus.

### 2. Description of the Related Art

In the related art, an ultrasound image showing a tomographic plane of a heart of a subject is captured using a so-called ultrasound diagnostic apparatus, and the ultrasound image is used to measure cardiac functions such as a so-called left ventricular ejection fraction (LVEF), a left ventricular end-diastolic volume, a left ventricular end-systolic volume, a left ventricular local wall motion, a global longitudinal strain (GLS), a mitral annular plane systolic excursion (MAPSE), a tricuspid annular plane systolic excursion (TAPSE), cardiac output, and stroke volume.

The measurement of the cardiac function may be performed based on a contour of an intracardiac cavity in the ultrasound image. It is known that the intracardiac cavity contour can be automatically extracted by analyzing ultrasound images, but, in a case in which the automatically extracted intracardiac cavity contour is erroneously extracted, or in a case in which the automatically extracted contour differs from the contour recognized by a user such as a doctor reviewing the ultrasound image, the extracted contour may not yield accurate or desired measured values of the cardiac function.

Therefore, for example, as disclosed in JP2018-507738A, a technique of correcting a contour of an intracardiac cavity that is automatically extracted has been developed. JP2018-507738A discloses that a user manually places a control point on the extracted contour of the intracardiac cavity, manually moves the placed control point, and corrects the contour in accordance with a position of the moved control point.

### SUMMARY OF THE INVENTION

However, in JP2018-507738A, since the user manually places the control point, it is difficult to place the control point at an appropriate position for correcting the contour to a desired shape and size, and the contour correction may take time. The measurement of the cardiac function is sometimes performed, for example, in emergency medical settings in which rapid treatment of a subject is required, and in a case in which the correction of the contour takes time, the measurement of the cardiac function of the subject and the functionality evaluation of the heart using the measured values cannot be carried out promptly, with the risk that prompt treatment of the subject will be impeded and other disadvantages will arise.

The present invention has been made in order to solve such a problem in the related art, and an object of the present invention is to provide an ultrasound diagnostic apparatus that can easily and accurately correct a contour of an intracardiac cavity and a method of controlling the ultrasound diagnostic apparatus.

It is possible to achieve the above object with the following configurations.

An ultrasound diagnostic apparatus comprising: an intracardiac cavity extraction unit that extracts an intracardiac cavity from an ultrasound image in which a heart of a subject is imaged; a correction possibility estimation unit that analyzes a contour of the intracardiac cavity extracted by the intracardiac cavity extraction unit and estimates a possibility of correction in each portion of the contour for evaluating functionality of the heart; a control point specifying unit that specifies a control point on the contour based on the possibility of correction estimated by the correction possibility estimation unit; an input device for a user to perform an input operation; a correction operation reception unit that receives a correction operation performed by the user via the input device on the control point specified by the control point specifying unit; and a contour correction unit that corrects the contour based on the correction operation received by the correction operation reception unit.

The ultrasound diagnostic apparatus according to [1], in which the correction possibility estimation unit estimates the possibility of correction in each portion of the contour based on a change in a shape of the contour over a plurality of heartbeats.

The ultrasound diagnostic apparatus according to [2], in which the control point specifying unit specifies the control point in a portion in which an amount of change in the shape of the contour over the plurality of heartbeats is equal to or greater than a first threshold value.

The ultrasound diagnostic apparatus according to [1], in which the intracardiac cavity extraction unit calculates a confidence level of a region of the extracted intracardiac cavity, and the correction possibility estimation unit estimates the possibility of correction in each portion of the contour based on the confidence level calculated by the intracardiac cavity extraction unit.

The ultrasound diagnostic apparatus according to [4], in which the control point specifying unit specifies the control point in a portion in which an amount of change in the confidence level in the region of the intracardiac cavity is equal to or less than a second threshold value.

The ultrasound diagnostic apparatus according to [1], in which the correction possibility estimation unit estimates the possibility of correction in each portion of the contour based on a change in a brightness value on the contour.

The ultrasound diagnostic apparatus according to [6], in which the control point specifying unit specifies the control point in a portion in which an amount of change in the brightness value on the contour is equal to or greater than a third threshold value.

A method of controlling an ultrasound diagnostic apparatus, the control method comprising: extracting an intracardiac cavity from an ultrasound image in which a heart of a subject is imaged; analyzing a contour of the extracted intracardiac cavity and estimating a possibility of correction in each portion of the contour for evaluating functionality of the heart; specifying a control point on the contour based on the estimated possibility of correction; receiving a correction operation performed by a user on the control point; and correcting the contour based on the correction operation.

The present invention provides the ultrasound diagnostic apparatus comprising: the intracardiac cavity extraction unit that extracts the intracardiac cavity from the ultrasound image in which the heart of the subject is imaged; the correction possibility estimation unit that analyzes the contour of the intracardiac cavity extracted by the intracardiac cavity extraction unit and estimates the possibility of correction in each portion of the contour for evaluating the functionality of the heart; the control point specifying unit that specifies the control point on the contour based on the possibility of correction estimated by the correction possibility estimation unit; the input device for the user to perform the input operation; the correction operation reception unit that receives the correction operation performed by the user via the input device on the control point specified by the control point specifying unit; and the contour correction unit that corrects the contour based on the correction operation received by the correction operation reception unit, so that it is possible to easily and accurately correct the contour of the intracardiac cavity.

### BRIEF DESCRIPTION OF THE DRAWINGS

FIG. 1 is a block diagram showing a configuration of an ultrasound diagnostic apparatus according to an embodiment of the present invention.

FIG. 2 is a block diagram showing an internal configuration of a transmission/reception circuit according to the embodiment of the present invention.

FIG. 3 is a block diagram showing an internal configuration of an image generation unit according to the embodiment of the present invention.

FIG. 4 is a schematic diagram showing an example of an ultrasound image showing an apical two-chamber cross section.

FIG. 5 is an example of a graph showing a time-series change in an area of an intracardiac cavity in the ultrasound image.

FIG. 6 is a diagram showing a plurality of contours of the intracardiac cavity corresponding to a plurality of end-diastolic phases of a heart in an overlaid manner.

FIG. 7 is a schematic diagram showing an example of a heat map of a confidence level of the intracardiac cavity.

FIG. 8 is an example of a graph showing a change in a brightness value of the ultrasound image on the contour of the intracardiac cavity.

FIG. 9 is a schematic diagram showing an example of a control point specified on the contour of the intracardiac cavity.

FIG. 10 is a flowchart showing an operation of the ultrasound diagnostic apparatus according to the embodiment of the present invention.

### DESCRIPTION OF THE PREFERRED EMBODIMENTS

Hereinafter, an embodiment of the present invention will be described with reference to the accompanying drawings.

The following configuration requirements are described based on a representative embodiment of the present invention, but the present invention is not limited to the embodiment.

In the present specification, a numerical range represented by "to" means a range including numerical values described before and after "to", both ends inclusive, as a lower limit value and an upper limit value.

In the present specification, "same" and "identical" include an error range which is generally allowed in the technical field.

### Embodiment

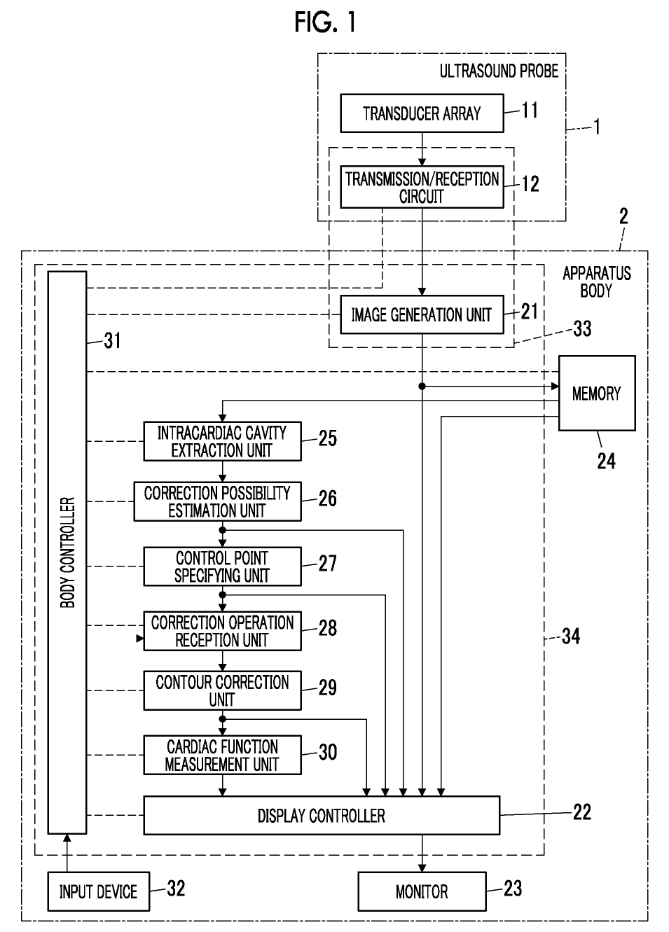

FIG. 1 shows a configuration of an ultrasound diagnostic apparatus according to the embodiment of the present invention. The ultrasound diagnostic apparatus comprises an ultrasound probe 1 and an apparatus body 2 that are connected to each other by so-called wired communication or so-called wireless communication.

The ultrasound probe 1 comprises a transducer array 11 and a transmission/reception circuit 12 connected to the transducer array 11.

The apparatus body 2 comprises an image generation unit 21 connected to the transmission/reception circuit 12. In the apparatus body 2, a display controller 22 and a monitor 23 are sequentially connected to the image generation unit 21. A memory 24 is connected to the image generation unit 21. An intracardiac cavity extraction unit 25 is connected to the memory 24. A correction possibility estimation unit 26, a control point specifying unit 27, a correction operation reception unit 28, a contour correction unit 29, and a cardiac function measurement unit 30 are sequentially connected to the intracardiac cavity extraction unit 25. The memory 24, the correction possibility estimation unit 26, the control point specifying unit 27, the contour correction unit 29, and the cardiac function measurement unit 30 are connected to the display controller 22. In addition, a body controller 31 is connected to the transmission/reception circuit 12, the image generation unit 21, the display controller 22, the memory 24, the intracardiac cavity extraction unit 25, the correction possibility estimation unit 26, the control point specifying unit 27, the correction operation reception unit 28, the contour correction unit 29, and the cardiac function measurement unit 30. An input device 32 is connected to the body controller 31.

In addition, the transmission/reception circuit 12 and the image generation unit 21 constitute an image acquisition unit 33. In addition, the image generation unit 21, the display controller 22, the intracardiac cavity extraction unit 25, the correction possibility estimation unit 26, the control point specifying unit 27, the correction operation reception unit 28, the contour correction unit 29, the cardiac function measurement unit 30, and the body controller 31 constitute a processor 34 for the apparatus body 2.

The transducer array 11 of the ultrasound probe 1 includes a plurality of ultrasound transducers that are one-dimensionally or two-dimensionally arranged. In accordance with a drive signal supplied from the transmission/reception circuit 12, each of the ultrasound transducers transmits ultrasound and receives an ultrasound echo from a subject to output a signal based on the ultrasound echo. Each of the ultrasound transducers is configured by forming, for example, electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 33 including the transmission/reception circuit 12 and the image generation unit 21 acquires an ultrasound image in which a heart of the subject is imaged by transmitting and receiving the ultrasound beams using the ultrasound probe 1.

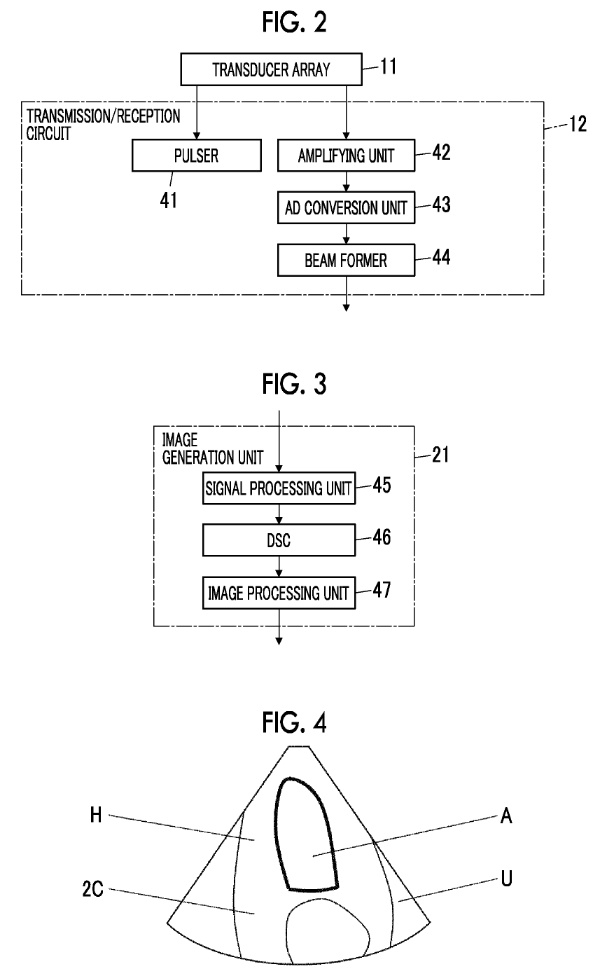

The transmission/reception circuit 12 transmits the ultrasound from the transducer array 11 and generates a beamformed signal based on the reception signal acquired by the transducer array 11, under the control of the body controller 31. As shown in FIG. 2, the transmission/reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, and the pulser 41 adjusts an amount of delay of each drive signal such that the ultrasound transmitted from the plurality of ultrasound transducers of the transducer array 11 form the ultrasound beams, based on a transmission delay pattern selected in accordance with a control signal from the body controller 31, and supplies the adjusted signal to the plurality of ultrasound transducers. In this way, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beams are formed from the combined wave of the ultrasound.

The transmitted ultrasound beam is, for example, reflected by a target such as a part of the subject and propagates toward the transducer array 11 of the ultrasound probe 1. The ultrasound echo propagating toward the transducer array 11 as described above is received by each of the ultrasound transducers constituting the transducer array 11. In such a case, each of the ultrasound transducers constituting the transducer array 11 receives the propagating ultrasound echo to expand and contract, generates the reception signal, which is an electrical signal, and outputs these reception signals to the amplifying unit 42.

The amplifying unit 42 amplifies the signal input from each of the ultrasound transducers constituting the transducer array 11, and transmits the amplified signal to the AD conversion unit 43. The AD conversion unit 43 converts the signal, which is transmitted from the amplifying unit 42, into digital reception data. The beam former 44 performs so-called receive focus processing by applying respective delays to the reception data received from the AD conversion unit 43 and then adding the delayed data together. By the receive focus processing, each reception data, which is converted by the AD conversion unit 43, is added in phase, and the beamformed signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 generates a B-mode image signal, which is tomographic image information related to tissues inside the subject, by performing, on the beamformed signal received from the transmission/reception circuit 12, correction of the attenuation due to a distance in accordance with a depth of a reflection position of the ultrasound by using a sound velocity value set by the body controller 31 and then performing envelope detection processing.

The DSC 46 converts (raster-converts) the B-mode image signal, which is generated by the signal processing unit 45, into the image signal in accordance with a standard television signal scanning method.

The image processing unit 47 performs various types of necessary image processing such as gradation processing on the B-mode image signal, which is input from the DSC 46, and then transmits the B-mode image signal to the display controller 22 and the memory 24. Hereinafter, the B-mode image signal, which is image-processed by the image processing unit 47, will be referred to as an ultrasound image.

The ultrasound diagnostic apparatus according to the embodiment of the present invention is used to evaluate the cardiac function by measuring cardiac functions such as a so-called left ventricular ejection fraction (LVEF), a left ventricular end-diastolic volume, a left ventricular end-systolic volume, a left ventricular local wall motion, a global longitudinal strain (GLS), a mitral annular plane systolic excursion (MAPSE), a tricuspid annular plane systolic excursion (TAPSE), cardiac output, and stroke volume.

In order to measure the cardiac function, for example, as shown in FIG. 4, an ultrasound image U showing an intracardiac cavity A, such as a so-called apical two-chamber cross section 2C of the heart H, is acquired by the image acquisition unit 33. The intracardiac cavity A means any one of a left ventricle, a left atrium, a right ventricle, or a right atrium. As the cross section of the intracardiac cavity A, in addition to the apical two-chamber cross section 2C, for example, a so-called apical three-chamber cross section, a so-called apical four-chamber cross section, a so-called apical five-chamber cross section, which are cross sections of the intracardiac cavity A passing through the cardiac apex, can also be imaged.

The display controller 22 performs predetermined processing on the ultrasound image U or the like acquired by the image acquisition unit 33, and displays the processed ultrasound image U or the like on the monitor 23, under the control of the body controller 31.

The monitor 23 displays the ultrasound image U or the like under the control of the display controller 22 and includes, for example, a display device such as a liquid crystal display (LCD) or an organic electroluminescence display (organic EL display).

The body controller 31 controls each unit of the apparatus body 2 and the transmission/reception circuit 12 of the ultrasound probe 1 based on a control program and the like stored in advance.

The input device 32 is an input device used by the user to perform an input operation and is configured by, for example, a device such as a keyboard, a mouse, a trackball, a touchpad, and a touch sensor disposed in a state of being superimposed on the monitor 23.

The memory 24 stores the ultrasound image U acquired by the image acquisition unit 33 under the control of the body controller 31.

As the memory 24, for example, a recording medium such as a flash memory, a hard disk drive (HDD), a solid-state drive (SSD), a flexible disk (FD), a magneto-optical disk (MO disk), a magnetic tape (MT), a random-access memory (RAM), a compact disc (CD), a digital versatile disc (DVD), a secure digital card (SD card), or a universal serial bus memory (USB memory) can be used.

The intracardiac cavity extraction unit 25 performs image analysis on the ultrasound image U acquired by the image acquisition unit 33 and stored in the memory 24 to extract the intracardiac cavity A from the ultrasound image U, for example, as shown in FIG. 4. In FIG. 4, the left ventricle of the heart H is shown as an example of the intracardiac cavity A.

The intracardiac cavity extraction unit 25 has, for example, a trained model in so-called machine learning, which has been trained in advance using a large number of ultrasound images U in which the intracardiac cavity A is imaged, and can output an extraction result of the intracardiac cavity A by inputting the ultrasound image U to the trained model.

In addition, the intracardiac cavity extraction unit 25 can also extract the intracardiac cavity A from the ultrasound image U by a so-called template matching method of storing, for example, a template image of the intracardiac cavity A in advance and searching for the ultrasound image U using the template image. In this way, the intracardiac cavity extraction unit 25 can extract the intracardiac cavity A from the ultrasound image U using various known methods.

The intracardiac cavity A extracted by the intracardiac cavity extraction unit 25 in this way may not be accurately extracted, for example, due to the insufficient image clarity of the ultrasound image U. In addition, the intracardiac cavity A extracted by the intracardiac cavity extraction unit 25 does not always coincide with a region of the intracardiac cavity A determined by the user by visually checking the ultrasound image U.

The correction possibility estimation unit 26 analyzes a contour of the intracardiac cavity A extracted by the intracardiac cavity extraction unit 25 and estimates a possibility of correction of each portion of the contour for evaluating the functionality of the heart H. Here, the possibility of correction estimated by the correction possibility estimation unit 26 includes a position at which there is a possibility of correction by the user such as a doctor on the contour of the intracardiac cavity A. In addition, the correction possibility estimation unit 26 estimates the possibility of correction with respect to the contour of the intracardiac cavity A in the ultrasound image U of a measurement frame used for measuring the cardiac function, for example, a frame selected by the user.

The correction possibility estimation unit 26 can estimate the possibility of correction in each portion of the contour for the ultrasound image U of the measurement frame, for example, based on a change in a shape of the contour of the intracardiac cavity A over a plurality of heartbeats. In this case, the correction possibility estimation unit 26 can estimate, for example, a portion in which an amount of change in the shape of the contour over the plurality of heartbeats is equal to or greater than a first threshold value, as a portion with the possibility of correction. Specific examples of such a method will be described below.

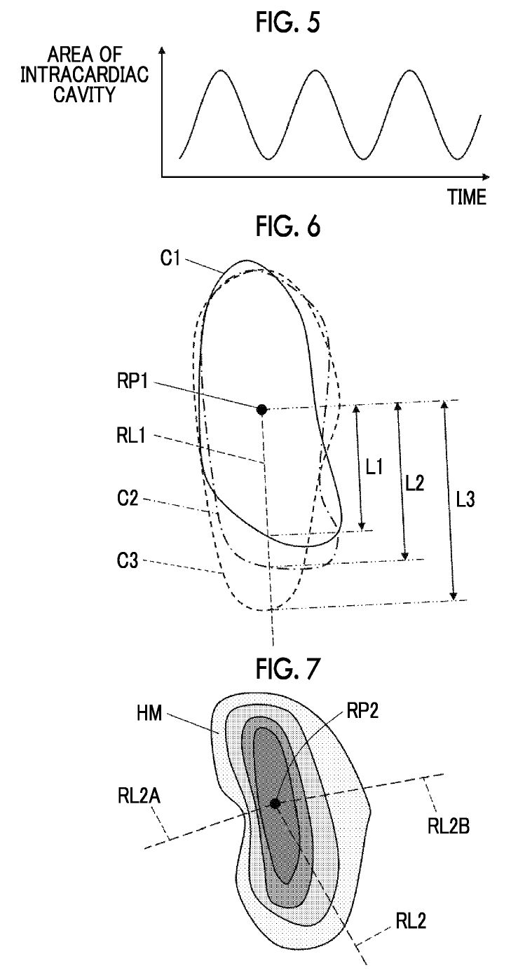

The correction possibility estimation unit 26 first calculates the areas of a plurality of intracardiac cavities A extracted by the intracardiac cavity extraction unit 25 in the ultrasound image U for the ultrasound images U of a plurality of continuous frames acquired by the image acquisition unit 33, and creates information indicating a correspondence relationship between the calculated values of the areas and the time points at which the ultrasound images U corresponding to the values of the respective areas are acquired. The information indicating the correspondence relationship can be created in a form of a graph as shown in FIG. 5, for example, or can be created in a form of a table. The correction possibility estimation unit 26 selects the intracardiac cavity A in the ultrasound image U of the frame at different heartbeats and the same time phase as the ultrasound image U of the measurement frame, with reference to the information indicating the correspondence relationship created in this way.

The correction possibility estimation unit 26 can calculate a total number of pixels present in the extracted region of the intracardiac cavity A, as the area of the intracardiac cavity A. In addition, as the ultrasound image U of the measurement frame, for example, the ultrasound image U corresponding to a so-called end-diastolic phase or a so-called end-systolic phase of the heart H can be selected. In general, the area of the intracardiac cavity A in the ultrasound image U is maximized in the end-diastolic phase, and the area of the intracardiac cavity A in the ultrasound image U is minimized in the end-systolic phase.

The correction possibility estimation unit 26 further overlays, for example, as shown in FIG. 6, a contour C1 of the intracardiac cavity A in the ultrasound image U of the measurement frame and contours C2 and C3 of the intracardiac cavity A in the ultrasound images U of the frames at different heartbeats and the same time phase as the ultrasound image U of the measurement frame, and sets a reference point RP1 inside the plurality of contours C1, C2, and C3. As the reference point RP1, for example, a centroid of the intracardiac cavity A in the ultrasound image U of the measurement frame can be set. The correction possibility estimation unit 26 sets a plurality of reference lines RL1 which are semi-straight lines extending radially from the reference point RP1, and calculates a distance L1 between the reference point RP1 and the contour C1, a distance L2 between the reference point RP1 and the contour C2, and a distance L3 between the reference point RP1 and the contour C3, in a direction along each reference line RL1.

The correction possibility estimation unit 26 calculates a distance [L2 - L1] between the contour C1 and the contour C2 and a distance [L3 - L1] between the contour C1 and the contour C3 in a direction along the reference line RL1. The correction possibility estimation unit 26 can determine that a portion in which the amount of change in the shapes of the contours C1, C2, and C3 over the plurality of heartbeats calculated in this way, that is, the distance [L2 - L1] or the distance [L3 - L1] is equal to or greater than the first threshold value is a portion in which the amount of change in the shapes of the contours C1, C2, and C3 is large, and can estimate the portion as the portion with the possibility of correction.

In addition, the intracardiac cavity extraction unit 25 can calculate a confidence level of the region of the extracted intracardiac cavity A in a case of extracting the intracardiac cavity A from the ultrasound image U. The confidence level represents a likelihood, that is, certainty, of the intracardiac cavity A of the extracted region and is calculated for each pixel of the ultrasound image U. The intracardiac cavity extraction unit 25 can calculate the confidence level in a so-called heat map HM format in the ultrasound image U, for example, as schematically shown in FIG. 7, by using a trained model in machine learning, which has been trained in advance using a relationship between the plurality of ultrasound images U showing the intracardiac cavity A and the confidence level for the pixels. The heat map HM is a diagram in which the ultrasound image U is colored in accordance with the magnitude of the confidence level, and can include contour lines of the confidence level as shown in FIG. 7.

In a case in which the intracardiac cavity extraction unit 25 calculates the confidence level of the region of the intracardiac cavity A, the correction possibility estimation unit 26 can also estimate the possibility of correction in each portion of the contour of the intracardiac cavity A based on the confidence level calculated by the intracardiac cavity extraction unit 25 for the ultrasound image U of the measurement frame. In this case, the correction possibility estimation unit 26 can estimate a portion in which the amount of change in the confidence level in the region of the intracardiac cavity A is equal to or less than a second threshold value, as the portion with the possibility of correction. Specific examples of such a method will be described below.

For example, as shown in FIG. 7, the correction possibility estimation unit 26 sets a reference point RP2 at a position at which the value of the confidence level is the highest in the heat map HM, and sets a plurality of reference lines RL2 which are semi-straight lines extending radially from the reference point RP2. The correction possibility estimation unit 26 calculates the amount of change, that is, a slope of the confidence level in the heat map HM along each reference line RL2.

In a case in which the slope of the confidence level is larger than a certain value, that is, in a case in which the confidence level is rapidly changed, for example, a portion in which the confidence level has begun to rapidly change on the reference line RL2A shown in FIG. 7 can be determined as the contour portion of the intracardiac cavity A. On the other hand, in a case in which the slope of the confidence level is equal to or less than a certain value, that is, in a case in which the confidence level is gradually changed, it is difficult to determine which portion on the reference line RL2B shown in FIG. 7 is the contour of the intracardiac cavity A, for example. Therefore, the correction possibility estimation unit 26 can estimate a portion in which the slope of the confidence level along the reference line RL2 is equal to or less than the second threshold value, as the portion with the possibility of correction.

In addition, the correction possibility estimation unit 26 can also estimate the possibility of correction in each portion of the contour based on a change in a brightness value on the contour of the intracardiac cavity A extracted by the intracardiac cavity extraction unit 25. In this case, the correction possibility estimation unit 26 can estimate a portion in which the amount of change in the brightness value on the contour of the intracardiac cavity A in the ultrasound image U of the measurement frame is equal to or greater than a third threshold value, as the portion with the possibility of correction. Specific examples of such a method will be described below.

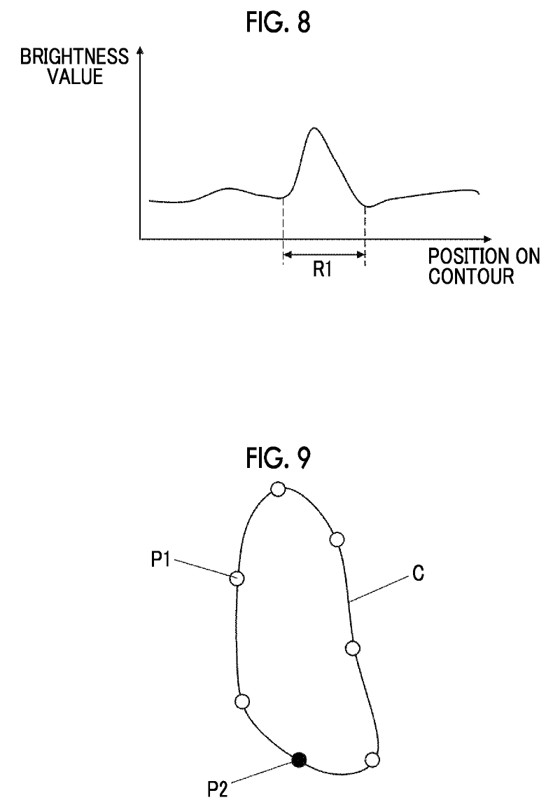

The correction possibility estimation unit 26 detects the brightness value on the contour of the intracardiac cavity A in the ultrasound image U of the measurement frame, and creates information indicating a correspondence relationship between a plurality of the detected brightness values and the position on the contour of the intracardiac cavity A. The information indicating the correspondence relationship can be created in a form of a graph as shown in FIG. 8, for example, or can be created in a form of a table. The position on the contour can be represented by, for example, a distance along the contour from the reference point to any point on the contour in a case in which the point on the contour of the intracardiac cavity A is set as the reference point.

Since the ultrasound image U shows the structure in the subject due to the change in the brightness value, the user, such as the doctor, usually determines the contour of the intracardiac cavity A by visually checking the ultrasound image U based on the brightness value of the pixel of the ultrasound image U. It is expected that the brightness values on the contour of the intracardiac cavity A determined by the user in this way are substantially the same. Therefore, in a case in which the amount of change in the brightness value on the contour of the intracardiac cavity A is equal to or greater than a certain value, it can be determined that the contour is detected on the structure that is not the contour of the intracardiac cavity A. Therefore, as shown in FIG. 8, the correction possibility estimation unit 26 can estimate a portion R1 in which the amount of change in the brightness value with respect to the position on the contour is equal to or greater than the third threshold value, as the portion with the possibility of correction.

The control point specifying unit 27 specifies a control point on the contour based on the possibility of correction estimated by the correction possibility estimation unit 26. The control point is for deforming the contour, and, in a case in which the user moves the control point via the input device 32, the contour can be deformed accordingly. For example, as shown in FIG. 9, the control point specifying unit 27 can specify a plurality of first control points P1 at a plurality of characteristic positions such as inflection points on the contour C or a plurality of positions at equal intervals, and then specify a second control point P2 at any position in the portion with the possibility of correction estimated by the correction possibility estimation unit 26.

The control point specifying unit 27 can specify the second control point P2 in a portion in which the amount of change in the shape of the contour C in the plurality of heartbeats is equal to or greater than the first threshold value, specify the second control point P2 in a portion in which the amount of change in the confidence level in the region of the intracardiac cavity A is equal to or less than the second threshold value, and specify the second control point P2 in a portion in which the amount of change in the brightness value on the contour C is equal to or greater than the third threshold value, in accordance with the method of estimating the possibility of correction by the correction possibility estimation unit 26.

The contour C, the first control point P1, and the second control point P2 are displayed on the monitor 23, for example, together with the ultrasound image U via the display controller 22.

The correction operation reception unit 28 receives a correction operation performed by the user via the input device 32 on the control point specified by the control point specifying unit 27. The correction operation specifically means an operation of moving the control point. The user moves the second control point P2 while checking the ultrasound image U, the contour C of the intracardiac cavity A, the first control point P1, and the second control point P2 that are displayed on the monitor 23. In this case, the user can finely adjust the shape of the contour C deformed by the movement of the second control point P2 by moving the first control point P1.

The display controller 22 can display the second control point P2 in a highlighted manner, for example, by displaying a display color of the second control point P2 in a color different from that of the first control point P1, so that the user can distinguish between the first control point P1 and the second control point P2. In addition, the display controller 22 can also perform highlight display, for example, by displaying the portion of the contour C of the intracardiac cavity A, which is estimated by the correction possibility estimation unit 26 to have the possibility of correction, in a color different from the colors of the other portions of the contour C, or the like.

The contour correction unit 29 corrects the contour C of the intracardiac cavity A based on the correction operation received by the correction operation reception unit 28, that is, in accordance with the positions of the first control point P1 and the second control point P2 moved by the user. The corrected contour C is displayed on the monitor 23 via the display controller 22.

Since the second control point P2 for deforming the portion with the possibility of correction, which is estimated by the correction possibility estimation unit 26, is automatically specified, and the contour C of the intracardiac cavity A can be corrected by moving the second control point P2 by the user, the user can easily and quickly obtain an accurate contour C of the intracardiac cavity A or a desired contour C.

The cardiac function measurement unit 30 measures the cardiac function of the subject based on the intracardiac cavity A in which the contour C is corrected by the contour correction unit 29. The cardiac function measurement unit 30 can measure, for example, a left ventricular ejection fraction, a left ventricular end-diastolic volume, a left ventricular end-systolic volume, a left ventricular local wall motion, a GLS, an MAPSE, a TAPSE, cardiac output, stroke volume, and the like, as the cardiac functions.

In a case of measuring, for example, the left ventricular ejection fraction as the cardiac function, the cardiac function measurement unit 30 calculates, for example, the volume of the left ventricle in the ultrasound image U extracted from the ultrasound image U showing the apical four-chamber cross section. In addition, the cardiac function measurement unit 30 similarly calculates the volume of the left ventricle extracted from the ultrasound image U showing the apical two-chamber cross section 2C. The cardiac function measurement unit 30 can measure the left ventricular ejection fraction based on the volume of the left ventricle in the ultrasound image U calculated for each cross section. In this case, the cardiac function measurement unit 30 can calculate the left ventricular ejection fraction by calculating the volume using, for example, a so-called modified-Simpson method, a stacked-disk method, an area-length method, and the like.

In the present embodiment, each processing is executed by any computer. In addition, any computer may execute these pieces of processing by the processor 34 as hardware, a program as software, or a combination thereof. In such a case, the processor 34 is configured to execute various types of processing in the present embodiment in cooperation with the program, and can function as each unit or each means in the present embodiment. In addition, the execution order of the processing by the processor 34 is not limited to the order described above and may be changed as appropriate. Any computer may be a general-purpose computer, a computer for a specific purpose, a workstation, or another system capable of executing the processing.

The processor 34 may be configured by one or more kinds of hardware, and the type of hardware is not limited. For example, the processor 34 may be implemented by hardware such as a programmable logic device, for example, a central processing unit (CPU), a micro processing unit (MPU), or a field programmable gate array (FPGA), a dedicated circuit for executing specific processing such as an application specific integrated circuit (ASIC), a graphics processing unit (GPU), or a neural processing unit (NPU). The types of hardware may be a combination of different types of hardware. In a case in which a plurality of types of hardware are configured to execute one or a plurality of types of processing of the processor 34, the plurality of types of hardware may be present in devices physically separated from each other or may be present in the same device. In any of the embodiments, the order of each processing performed by the processor 34 is not limited to the above-described order, and may be changed as appropriate. Hardware is implemented in a form of an electric circuit (circuitry) in which circuit elements such as semiconductor elements are combined.

Furthermore, the program may be software, such as firmware or a microcode. Moreover, the program may be, for example, a program module group, and each function thereof may be implemented by the processor 34 configured to execute each function. The program may be a program code or a plurality of code segments stored in one or a plurality of non-transitory computer-readable media (for example, a storage medium or another storage). The program may be stored in a plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

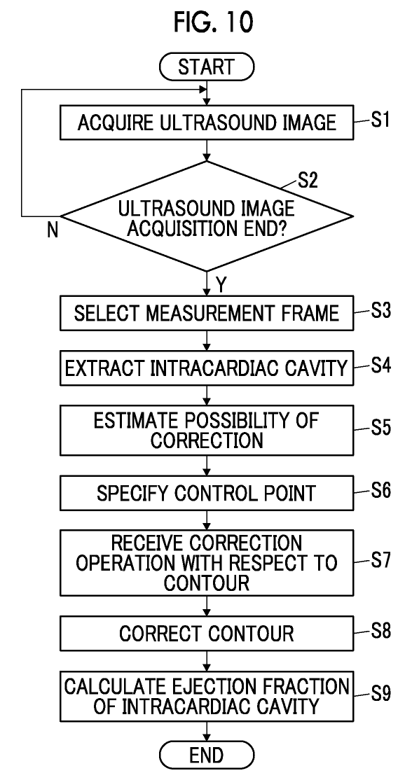

Hereinafter, an operation of the ultrasound diagnostic apparatus according to the embodiment will be described with reference to a flowchart shown in FIG. 10.

First, in step S1, the user disposes the ultrasound probe 1 at a position for capturing the ultrasound image U showing a desired cross section used for measuring the cardiac function. The image acquisition unit 33 acquires the ultrasound image U including the intracardiac cavity A. In such a case, under the control of the body controller 31, the transmission and reception of the ultrasound from the plurality of transducers of the transducer array 11 are started in accordance with the drive signal from the pulser 41 of the transmission/reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The receive focus processing is performed on the reception data by the beam former 44, the beamformed signal generated by the reception focusing processing is transmitted to the image generation unit 21 of the apparatus body 2, and thus the ultrasound image U is generated by the image generation unit 21. In such a case, the signal processing unit 45 of the image generation unit 21 performs the correction of the attenuation in accordance with the depth of the reflection position of the ultrasound and the envelope detection processing on the beamformed signal, the DSC 46 performs the conversion into the image signal in accordance with the normal television signal scanning method, and the image processing unit 47 performs various types of necessary image processing, such as gradation processing. The ultrasound image U generated in step S1 in this way is sent to the display controller 22 and the memory 24.

In step S2, the body controller 31 determines whether or not to end the acquisition of the ultrasound image U. The body controller 31 can determine that the acquisition of the ultrasound image U should end, for example, in a case in which the instruction to end the acquisition of the ultrasound image U is input by the user via the input device 32. In addition, the body controller 31 can determine that the acquisition of the ultrasound image U should be continued, for example, in a case in which an instruction to end the acquisition of the ultrasound image U is not particularly input from the user via the input device 32. While it is determined that the acquisition of the ultrasound image U should be continued, the processing of step S1 and step S2 is repeated. As a result, the ultrasound images U of the plurality of frames are stored in the memory 24. In a case in which the user inputs the instruction via the input device 32 by determining that the ultrasound image U is sufficiently acquired or the like, and it is determined that the acquisition of the ultrasound image U should end, the processing proceeds to step S3.

In step S3, the body controller 31 selects the ultrasound image U of the measurement frame used for measuring the cardiac function from among the ultrasound images U of the plurality of frames stored in the memory 24 in step S1. The body controller 31 can select the ultrasound image U of the measurement frame, for example, in response to the instruction from the user via the input device 32. In this case, for example, the body controller 31 displays the ultrasound images U of the plurality of frames stored in the memory 24 on the monitor 23 based on the instruction of the user, and the user can designate the ultrasound image U of one frame of the plurality of frames of the ultrasound images U displayed on the monitor 23, as the ultrasound image U of the measurement frame.

In step S4, the intracardiac cavity extraction unit 25 extracts the intracardiac cavity A from the ultrasound image U by performing the image analysis on the ultrasound image U of the measurement frame selected in step S3. The intracardiac cavity extraction unit 25 can extract the intracardiac cavity A from the ultrasound image U by, for example, a method of using a trained model in machine learning, which has been trained using a large number of ultrasound images U in which the intracardiac cavity A is imaged, a template matching method, or the like.

The intracardiac cavity extraction unit 25 can extract the intracardiac cavity A from the ultrasound images U of the plurality of frames other than the ultrasound image U of the measurement frame, depending on the processing method of the correction possibility estimation unit 26 in the next step S5. In addition, the intracardiac cavity extraction unit 25 can also calculate the confidence level of the extracted intracardiac cavity A depending on the processing method of the correction possibility estimation unit 26 in the next step S5. The confidence level is calculated for each pixel in the form of the heat map HM as shown in FIG. 7, for example.

In step S5, the correction possibility estimation unit 26 analyzes the contour C of the intracardiac cavity A extracted in step S4 and estimates the possibility of correction of each portion of the contour C for evaluating the functionality of the heart H.

For example, in a case in which the intracardiac cavities A are extracted from the ultrasound images U of the plurality of frames in step S4, the correction possibility estimation unit 26 can estimate the possibility of correction in each portion of the contour C based on the change in the shape of the contour C over the plurality of heartbeats. In this case, the correction possibility estimation unit 26 calculates, for example, the areas of the plurality of extracted intracardiac cavities A, and creates information indicating the correspondence relationship between the values of the plurality of calculated areas and the acquisition time points of the ultrasound images U including the respective intracardiac cavities A, as shown in FIG. 5. The correction possibility estimation unit 26 specifies the ultrasound image U at different heartbeats and the same time phase as the ultrasound image U of the measurement frame selected in step S3 with reference to the information indicating the correspondence relationship.

For example, as shown in FIG. 6, the correction possibility estimation unit 26 sets the reference point RP1 inside the intracardiac cavity A in the ultrasound image U of the measurement frame by overlaying the contour of the intracardiac cavity A in the ultrasound image U of the measurement frame and that in the ultrasound image U of the specified frame. The reference point RP1 can be set to, for example, the centroid position of the intracardiac cavity A in the ultrasound image U of the measurement frame.

The correction possibility estimation unit 26 can set the plurality of reference lines RL1 which are straight lines extending radially from the reference point RP1, calculate the distances [L2 - L1] and [L3 - L1] between the contour C1 of the intracardiac cavity A in the ultrasound image U of the measurement frame and the other plurality of intracardiac cavities A in a direction along each reference line RL1, and in a case in which the calculated distance [L2 - L1] or [L3 - L1] is equal to or greater than the first threshold value, estimate a portion on the contour C1 corresponding to the reference line RL1 as the portion with the possibility of correction. In this way, the correction possibility estimation unit 26 can estimate a portion that may be erroneously extracted as the contour C of the intracardiac cavity A.

In a case in which the confidence level of the intracardiac cavity A extracted by the intracardiac cavity extraction unit 25 is calculated for the ultrasound image U of the measurement frame in step S4, the correction possibility estimation unit 26 can also estimate the possibility of correction in each portion of the contour C based on the confidence level.

For example, as shown in FIG. 7, the correction possibility estimation unit 26 sets the reference point RP2 at a position at which the confidence level is highest in the heat map HM of the confidence level, and sets the plurality of reference lines RL2 which are semi-straight lines extending radially from the reference point RP2. The correction possibility estimation unit 26 can calculate the amount of change, that is, the slope of the confidence level along each reference line RL2, and calculate a portion in which the calculated slope is equal to or less than the second threshold value as the portion with the possibility of correction. In this way, the correction possibility estimation unit 26 can estimate a portion that may be erroneously extracted as the contour C of the intracardiac cavity A.

The correction possibility estimation unit 26 can also estimate the possibility of correction in each portion of the contour C based on the change in the brightness value on the contour C of the intracardiac cavity A in the ultrasound image U of the measurement frame extracted in step S4. The correction possibility estimation unit 26 can calculate, for example, a portion in which the amount of change in the brightness value with respect to the position on the contour C of the intracardiac cavity A is equal to or greater than the third threshold value, as the portion with the possibility of correction. In this way, the correction possibility estimation unit 26 can estimate a portion that may be different from the contour C determined by the user by visually checking the ultrasound image U in the extracted contour C of the intracardiac cavity A.

In step S6, the control point specifying unit 27 specifies the plurality of control points on the contour C based on the possibility of correction of the contour C estimated in step S5. For example, as shown in FIG. 9, the control point specifying unit 27 can specify the plurality of first control points P1 at the plurality of characteristic positions such as inflection points of the contour C or the plurality of positions at equal intervals on the contour C, and can specify the second control point P2 at any position in the portion with the possibility of correction on the contour C, which is estimated in step S5.

In step S7, the correction operation reception unit 28 receives the correction operation performed by the user via the input device 32 on the first control point P1 and the second control point P2 that are specified in step S6. In this case, the user can select the first control point P1 or the second control point P2 via the input device 32, and move the selected first control point P1 or second control point P2 on the ultrasound image U. In a case in which any of the first control point P1 or the second control point P2 is moved, the contour C is deformed accordingly.

In this case, the display controller 22 can display the second control point P2 specified at a position with the possibility of correction on the monitor 23 in a highlighted manner by changing the display color or the like so that the user can recognize the second control point P2 distinguished from the first control point P1. By moving the second control point P2, the user can obtain an accurate contour C of the intracardiac cavity A or a desired contour C of the user.

In step S8, the contour correction unit 29 corrects the contour C of the intracardiac cavity A based on the correction operation by the user received in step S7.

Finally, in step S9, the cardiac function measurement unit 30 measures the cardiac function such as the left ventricular ejection fraction by using the contour C of the intracardiac cavity A corrected in step S8. The measured value of the cardiac function obtained in this manner is displayed on, for example, the monitor 23 and is used for the evaluation of the functionality of the heart H by the user such as the doctor.

In a case in which the processing of step S9 is completed in this manner, the operation of the ultrasound diagnostic apparatus represented by the flowchart shown in FIG. 10 is completed.

As described above, with the ultrasound diagnostic apparatus according to the embodiment of the present invention, the intracardiac cavity extraction unit 25 extracts the intracardiac cavity A from the ultrasound image U in which the heart H of the subject is imaged, the correction possibility estimation unit 26 analyzes the contour C of the extracted intracardiac cavity A and estimates the possibility of correction in each portion of the contour C, the control point specifying unit 27 specifies the control point on the contour C of the intracardiac cavity A based on the estimated possibility of correction, the correction operation reception unit 28 receives the correction operation performed by the user on the specified control point, and the contour correction unit 29 corrects the contour C based on the received correction operation, so that the contour C of the intracardiac cavity A can be easily and accurately corrected.

The configuration has been described in which the transmission/reception circuit 12 is provided in the ultrasound probe 1, but the transmission/reception circuit 12 may be provided in the apparatus body 2.

Further, the configuration has been described in which the image generation unit 21 is provided in the apparatus body 2, but the image generation unit 21 may be provided in the ultrasound probe 1.

The apparatus body 2 may be a so-called stationary type, a portable type that is easily carried, or a so-called handheld type that is configured by, for example, a smartphone or a tablet type computer. As described above, the types of the devices constituting the apparatus body 2 are not particularly limited.

Although the example has been described in which the control point specifying unit 27 specifies the plurality of first control points P1 at the plurality of characteristic positions in the contour C of the intracardiac cavity A or at the plurality of positions at equal intervals on the contour C, the position of the first control point P1 is not particularly limited thereto. For example, the control point specifying unit 27 can determine that a portion of the contour C of the intracardiac cavity A, which is not the portion with the possibility of correction estimated by the correction possibility estimation unit 26, is a portion having a high possibility of representing an accurate contour C and is not preferable to be deformed, and specify the first control points P1 at a plurality of positions in addition to the plurality of characteristic positions in the contour C.

In addition, in step S3 of the flowchart of FIG. 10, the example has been described in which the user manually selects the ultrasound image U of the measurement frame, but the method of selecting the ultrasound image U of the measurement frame is not particularly limited to this. For example, after the intracardiac cavities A are extracted from the ultrasound images U of the plurality of frames stored in the memory 24 in step S4, the body controller 31 can calculate the areas of the plurality of intracardiac cavities A, create information indicating a correspondence relationship between the values of the plurality of calculated areas and the acquisition time points of the ultrasound images U, and select, for example, the ultrasound image U corresponding to a specific time phase such as the end-diastolic phase or the end-systolic phase of the heart H as the ultrasound image U of the measurement frame, based on the created information indicating the correspondence relationship.

### Explanation of References

1: ultrasound probe

2: apparatus body

11: transducer array

12: transmission/reception circuit

21: image generation unit

22: display controller

23: monitor

24: memory

25: intracardiac cavity extraction unit

26: correction possibility estimation unit

27: control point specifying unit

28: correction operation reception unit

29: contour correction unit

30: cardiac function measurement unit

31: body controller

32: input device

33: image acquisition unit

34: processor

41: pulser

42: amplifying unit

43: AD conversion unit

44: beam former

45: signal processing unit

46: DSC

47: image processing unit

2C: apical two-chamber cross section

A: intracardiac cavity

C, C1, C2, C3: contour

L1, L2, L3: distance

H: heart

HM: heat map

P1: first control point

P2: second control point

R1: portion

RL1, RL2, RL2A, RL2B: reference line

RP1, RP2: reference point

U: ultrasound image

What is claimed is:

1. An ultrasound diagnostic apparatus comprising:
a processor configured to:
extract an intracardiac cavity from an ultrasound image in which a heart of a subject is imaged;
analyze a contour of the intracardiac cavity and estimate a possibility of correction in each portion of the contour for evaluating functionality of the heart;
specify a control point on the contour based on the possibility of correction;
receive a correction operation performed by a user on the control point; and
correct the contour based on the correction operation.

2. The ultrasound diagnostic apparatus according to claim 1,
wherein the processor is configured to estimate the possibility of correction in each portion of the contour based on a change in a shape of the contour over a plurality of heartbeats.

3. The ultrasound diagnostic apparatus according to claim 2,
wherein the processor is configured to specify the control point in a portion in which an amount of change in the shape of the contour over the plurality of heartbeats is equal to or greater than a first threshold value.

4. The ultrasound diagnostic apparatus according to claim 1,
wherein the processor is configured to:
calculate a confidence level of a region of the extracted intracardiac cavity; and
estimate the possibility of correction in each portion of the contour based on the confidence level.

5. The ultrasound diagnostic apparatus according to claim 4,
wherein the processor is configured to specify the control point in a portion in which an amount of change in the confidence level in the region of the intracardiac cavity is equal to or less than a second threshold value.

6. The ultrasound diagnostic apparatus according to claim 1,
wherein the processor is configured to estimate the possibility of correction in each portion of the contour based on a change in a brightness value on the contour.

7. The ultrasound diagnostic apparatus according to claim 6,
wherein the processor is configured to specify the control point in a portion in which an amount of change in the brightness value on the contour is equal to or greater than a third threshold value.

8. A method of controlling an ultrasound diagnostic apparatus, the control method comprising:
extracting an intracardiac cavity from an ultrasound image in which a heart of a subject is imaged;
analyzing a contour of the extracted intracardiac cavity and estimating a possibility of correction in each portion of the contour for evaluating functionality of the heart;
specifying a control point on the contour based on the estimated possibility of correction;
receiving a correction operation performed by a user on the control point; and
correcting the contour based on the correction operation.
